(19) United States
(12) Patent Application Publication
(10) Pub. No.: US 2010/0312324 A1
(43) Pub. Date: Dec. 9, 2010

(54) IMPLANTATION DEVICE FOR STENTS WITH A FUNCTIONALLY STRUCTURED SURFACE

(76) Inventors: Nina Adden, Nuernberg (DE); Bettina Surber, Gachnang (CH)

Correspondence Address: GREER, BURNS & CRAIN, 300 S WACKER DR, 25TH FLOOR, CHICAGO, IL, 60606, US

(21) Appl. No.: 12/774,931

(22) Filed: May 6, 2010

Related U.S. Application Data
(60) Provisional application No. 61/183,991, filed on Jun. 4, 2009.
(60) Provisional application No. 61/184,321, filed on Jun. 5, 2009.

(51) Int. Cl. A61F 2/84

(52) U.S. Cl. 623/1.12

(57) ABSTRACT

The invention relates to an implantation device for stents, which device has hair-like extensions on at least one part of the outwardly-facing surface of that section of the device, which section is provided to attach or crimp on the stent to be implanted, wherein the mean diameter D, the mean length L, and the averaged center-to-center distance P of the hair-like extensions relative to each other are selected such that a stent is affixable on the surface covered with hair-like extensions essentially by means of van der Waals forces.

### FIELD OF THE INVENTION

This invention relates to the field of implantation devices for stents.

### BACKGROUND

Implantation of stents has become established as one of the most effective therapeutic measures in the treatment of vascular diseases. The purpose of stents is to take on a support function within the hollow organs of a patient. To this end, conventionally-designed stents have a tubular base body with a filigree support structure composed of metallic struts, the support body first being provided in a compressed form for insertion into the body and then expanded at the site of implantation. One of the principal areas of application for these stents is the permanent or temporary widening and keeping-open of vascular constrictions, in particular, constrictions (stenoses) of the coronary blood vessels. In addition, for example, aneurysm stents are also known that function to support the damaged vascular walls.

Stents have a circumferential wall of sufficient load-bearing capacity to keep the narrowed blood vessel open in the desired degree, as well as a tubular base body through which the flow of blood can continue to move unobstructed. The circumferential wall is typically composed of a lattice-like supporting structure that allows the stent to be inserted in a compressed state of small outer diameter up to the constriction site of the specific vessel to be treated where it is implanted—for example, by means of an implantation device, for example, a catheter—in such a way that the vessel has the desired enlarged interior diameter.

Various implantation devices can be employed for implantation of the stent.

Generally balloon catheters are used for the implantation of non-self-expanding stents. Here the stent is affixed to the surface of the nondilated balloon, typically by what is known as “crimping.” Once the balloon catheter loaded with the stent has been inserted into the vessel and the stent has reached the site at which it is to be implanted, the balloon is dilated. As a result, the stent is expanded and brought into contact with the vascular wall. After the stent has been sufficiently expanded and the vessel has been sufficiently dilated, the balloon returns to a nondilated state and can be removed while the implanted stent remains in position within the vessel and keeps it open. Balloon catheters suitable for implantation of stents are well known and described, for example, in DE 102 15 462.

Alternatively, a self-expanding stent design may be used. Implantation devices for the implantation of self-expanding stents generally do not have a dilatable balloon to effect implantation of the stent. In this case, catheter devices are typically used that have an interior tube and exterior tube, where the stent to be implanted is initially provided in a crimped state on one surface of the interior tube and is covered by an exterior tube that has been slid thereover. Self-expanding stents in the crimped form exhibit a radial force that presses the outer surface of the stent against the inner surface of the exterior tube, the inner surface covering the stent. The catheter is inserted into the vessel until the stent is situated at the site at which it is to be implanted. In order to effect the implantation, the exterior tube is now retracted until the entire stent is exposed. In response to the radial force, the stent expands automatically while the implanted stent remains in position in the vessel and keeps this open. Balloon catheters suitable for implantation of self-expanding stents are well known and are described, for example, in U.S. Pat. No. 5,824,041.

What is critical both for the implantation of balloon catheters as well as the implantation of self-expanding stents is that a sufficient retention force be created between the crimped stent and the surface of the implantation device on which the stent is situated in the crimped state.

In particular, a self-expanding stent must be affixed on the interior tube in such a way that it does not slip during the implantation procedure when the exterior tube is removed.

In the prior art, this is achieved by using a so-called stent stopper that is attached proximally to the stent on the interior tube. In this solution, however, the stent is pushed together into itself by an increase in friction when the stent is released, an action that can impair the properties of the stent.

In the case of implantation by means of the balloon catheter, the stent must be provided in fixed form on the nondilated surface of the balloon. This is not always possible to a sufficient degree for all types of stents. For example, pharmaceutical-agent-releasing stents, stents having a large strut wall thickness, or stents having a large strut width can often not be affixed sufficiently by simple crimping.

Up until now, it is been necessary in such cases to implement an additional fixation means of the stent that must be superimposed after the fact on the crimped stent—a measure that generally is not possible by means of a machine. In addition, these stent fixation means can impair the expansion behavior of the stent and/or damage the coating of a coated stent.

The problem to be solved by this invention is to mitigate or prevent one or more of the disadvantages of the prior art. In particular, the goal is to provide implantation devices for stents that require fewer additional measures for fixation of the stent.

### SUMMARY

The problem is solved by providing an implantation device for stents that have hair-like extensions on at least one part of the outward-facing surface of that section of the device, which section is provided for attaching or crimping the stent to be implanted, wherein the mean diameter D, mean length L, and averaged center-to-center distance P of the hair-like extensions relative to each other are selected such that a stent is essentially affixable by van der Waals forces on the surface covered with hair-like extensions.

Based on the surface structure of the inner surface of Gecko feet, a surface of the implantation device has a plurality of hair-like extensions. Due to a multitude of submicrometer-sized tiny hairs on its feet, the gecko is able to walk vertically or also upside-down even on smooth surfaces. This effect is a combination of capillary forces and van der Waals forces. In the implantation device according to the invention, it is specifically the van der Waals is forces of this type of surface structure that are used to affix a crimped stent on a surface of the implantation device and then to release it as required. This surface structure enables the measures following crimping used to effect stent fixation to be eliminated. As a result of the contact between the referenced surface structure of the implantation device and the inner surface of the stent, the stent is essentially affixed on the implantation device due to van der Waals forces. The sum of van der Waals forces per hair-like extension together produces sufficient retention force for the stent. The van der Waals forces here between the inner surface of the stent and the hair-like extensions are greatest whenever the force acts vertically on the contact area of the hair-like extensions, such as when a balloon catheter provided with a stent is introduced into a vessel. No longer is an elaborate stent fixation procedure required to attach a stent on the implantation device after crimping.

In principle, any implantation device for stents can be employed, provided that a surface of the stent to be implanted is in contact before implantation with a surface for the implantation device. Preferably, the implantation device comprises a catheter.

The implantation device can comprise a dilatable balloon surface onto which a stent can be crimped, such as, for example, one having a balloon catheter. In principle, any known balloon catheter system can be used for the balloon catheter according to the invention. In particular, a balloon catheter can be used having an inner shaft to which one distal end of an expanding balloon is attached which in a nonexpanded deflated state at least partially contacts an outer surface of the inner shaft. The invention relates in particular to those catheters that can support a stent on the outside of the deflated balloon, which stent after insertion into the vessel is expanded by inflating the balloon with a fluid and thereby pressed against a vascular wall at the intended site.

In addition to an inner shaft and the balloon, balloon catheters of the type provided typically also have an outer shaft that extends at least up to the proximal end of the balloon and is attached to this balloon in a fluid-tight manner. Typically, a fluid conduit is provided extending in the longitudinal axis of the catheter from its proximal end into the interior of the balloon, which conduit is created, for example, due to the fact that the outer shaft has an inside diameter that is larger than an outside diameter of the inner shaft.

Provided inside the inner shaft is a cavity enclosed by the inner shaft, extending longitudinally relative to the inner shaft, and functioning as the lumen. This lumen functions, for example, to receive a mandrin or a guide wire. Catheter and guide wire are then designed, for example, such that the guide wire can emerge from the distal tip of the catheter and can be controlled from the proximal end. The guide wire is, for example, deflected by control means such that it can easily be introduced into branching blood vessels. The balloon catheter can then be advanced along the guide wire.

Regardless of the type of catheter, specifically in terms of the design of the guide means, balloon catheters have the above-referenced expandable balloon at their distal end. The balloon is compressed during insertion of the balloon catheter and rests tightly against the inner shaft of the catheter. The catheter can be expanded by inflating the balloon with a fluid. This expansion occurs as soon as the balloon has been guided up to the intended position. The stent is plastically deformed by the expansion of the balloon, thereby enabling it to attach permanently to the vascular wall.

Alternatively, the implantation device according to the invention can be designed as a device for the implantation of self-expanding stents, for example, can have a surface for implantation of the self-expanding stent. Devices for implantation of self-expanding stents are characterized in that no means are required for active plastic deformation of a crimped-on stent. Preferably, catheters are used that instead of a balloon have a section of an inner shaft onto which the self-expanding stent can be crimped to effect the implantation. In addition, this type of catheter has means that allow the crimped self-expanding stent to be prevented from self-expanding until the stent has been moved into the specified position and then allow self-expansion of the stent to be triggered. This can be achieved, for example, by surrounding the self-expanding stent that is crimped on the interior tube with an exterior tube which prevents the stent from self-expanding. This exterior tube can be designed such that retraction of the exterior tube relative to the interior tube is possible, thereby enabling a section of the interior tube to be exposed on which the self-expanding stent is located in a crimped state. Once the exterior tube has been retracted to a sufficient degree, the stent due to its radial force can detach itself from the interior tube, expand automatically, and move into contact with the vascular wall.

On at least one part of the outwardly-facing surface of the section of the device, the implantation device according to the invention has hair-like extensions, which section is provided for crimping the stent to be implanted. In particular, these sections of the implantation device, or portions thereof, can be provided with hair-like extensions that before an implantation are in contact with a surface of the stent to be implanted. Preferably, at least 20% of the surface coverable by the stent to be implanted has hair-like extensions, especially preferably 30% to 100% of the surface coverable by the stent to be implanted has hair-like extensions. The hair-like extensions are of a shape, the end of which rests on the surface of the implantation device and which rises above this surface. In particular, the hair-like extensions can have one or more lateral surfaces, and optionally a top surface, which faces away from the surface of the implantation shape and which can be brought into contact with one surface of the stent to be implanted. What is preferred are hair-like extensions that are essentially cylindrical.

The hair-like extensions can in principle be fabricated out of any material which can be attached to a surface of the implantation device, the surface being coverable by the stent to be implanted, and which is sufficiently compatible for use within the human body. The hair-like extensions preferably are composed of materials or combinations of material that are of a strength and stiffness such that the hair-like extensions can project vertically from the surface and do not collapse into themselves or buckle. In particular, the hair-like extensions can be fabricated out of the same material from which the surface of the implantation device is fabricated, which surface is coverable by the stent to be implanted. Appropriate materials and polymers are familiar to the person skilled in the art.

A surface having hair-like extensions can be produced by a variety of methods. For example, it is possible to generate negative molds by lithographic methods such as, e.g., electron beam lithography and laser lithography, or by etching techniques. Starting with the negative mold, the positive surface having hair-like extensions is then generated in a subsequent casting process (see, for example, A. K. Geim et al., Nature Mater. 2, 461-463 (2003), and H. Lee, B. P. Lee and P. B. Messersmith, Nature 448, 338-341 (2007)).

The mean diameter D of the hair-like extensions, the mean length L of the hair-like extensions, and the averaged center-to-center distance P of the hair-like extensions relative to each other are parameters that affect the magnitude of the attainable van der Waals forces.

D can be determined by determining the diameters of all hair-like extensions for a selected area, adding these together, and dividing the obtained total by the number of measured hair-like extensions.

L can be determined by determining the lengths of all hair-like extensions for a selected area, adding these together, and dividing the obtained total by the number of measured hair-like extensions.

P can be determined by determining the distances of all hair-like extensions relative to the respective closest hair-like extension, where measurement is effected starting from a center of a first hair-like extension from the center of the closest hair-like extension. The distance values obtained are added together, and the obtained total is divided by the number of measured hair-like extensions.

According to the invention, D, L, and P are selected such that a specific stent is affixable on the surface of the implantation device, the surface being covered by hair-like extensions. The material properties of the materials used to produce the hair-like extensions also affect the achievable van der Waals forces, for example, the density of the material surface. The required van der Waals forces needed to affix a specific stent according to the invention to the surface of the implantation device are not identical for every stent and must therefore be re-determined for each combination of stent and implantation device according to the invention.

The person skilled in the art can easily use routine tests to determine a combination of D, L, and P that is suitable for affixing to the desired degree a specific stent to the surface of is the implantation device, which surface is covered with hair-like extensions. For this purpose, test surfaces can be prepared that have hair-like extensions with various combinations of D, L, and P. These test surfaces can then be brought into contact with the surface sections of the desired stent, or with the inner surfaces of entire stents. The force is then measured which is required to remove the test surface from the stent surface.

In particular, D can be selected from a range between 0.05 and 5 μm, preferably, 0.1 and 4 μm, especially preferably 0.2 and 1 μm. L can, in particular, be selected from a range between 0.1 and 20 μm, preferably, 0.1 and 5 μm, especially preferably 0.15 μm and 2 μm. In particular, P can be selected from a range between 0.5 and 5 μm, preferably, between 0.5 and 3 μm. D and L are preferably selected so as to yield a ratio of D to L that is selected from a range between 0.5 and 2, preferably, 0.75 and 1.5. Preferably, P and D are selected so as to yield a ratio of P to D that is selected from a range between 1 and 50, preferably, 1.5 and 10.

The implantation device according to the invention can have hair-like extensions, wherein the hair-like extensions are coated with a polymer having catechol side groups. These polymers enhance the adhesiveness of the hair-like extensions. Suitable polymers as well as polymer compositions and methods to effect coating have been disclosed in WO 08/091,386. These polymers can have catecholamines, such as dopamine and the amino acid 3,4-dihydroxy-L-phenylalanine, also known as DOPA. An especially preferred polymer is poly(dopaminemethacrylamide (DMA)-co-methoxyethylacrylate (MEA)). Preferred polymers have catechol side groups, where catechol side groups constitute at least 5 wt. % of the total polymer weight, especially preferably, from 10 wt. % to 70 wt. %. The coating of the hair-like extensions with a polymer of the catechol side groups can have a coating thickness of less than 100 nm; preferably, the coating thickness is selected from a range between 1 nm and 50 nm.

This invention relates to an implantation device that additionally has an implantable stent, an implantable, pharmaceutical-agent-releasing stent, or an implantable biodegradable stent. The stent is preferably attached to the implantation device in such a way that the stent is implantable. In particular, the stent is attached to or crimped onto a surface of the implantation device, which surface is equipped with hair-like extensions provided for this purpose and in a manner at least partially described above. What is understood by biodegradable is, in particular, stents that have biocorrodible alloys of the elements, magnesium, iron, or tungsten for the base body of the stent. The composition of the alloys is selected so as to be biocorrodible. Those alloys are defined as biocorrodible according to the invention for which within a physiological environment a decomposition takes place that ultimately causes the entire stent, or the part of the stent composed of this material, to loose its mechanical integrity. One test medium for the purpose of testing the corrosion behavior of a possible alloy to be considered is an artificial plasma such as that specified by EN ISO 10993-15:2000 for biocorrosion analyses (composition NaCl 6.8 g/l, CaCl2 0.2 g/l, KCl 0.4 g/l, MgSO4 0.1 g/l, NaHCO3 2.2 g/l, Na2HPO4 0.126 g/l, NaH2PO4 0.026 g/l). A sample of the alloy to be analyzed is stored in a closed sample container with a defined test quantity of the test medium at 37° C. At certain time intervals—matched to the corrosion behavior expected—ranging from a few hours up to several months, the samples are removed and analyzed in the known manner for traces of corrosion. The artificial plasma according to EN ISO 10993-15:2000 corresponds to a blood-like medium and provides a means of reproducing a physiological environment as defined by the invention.

This invention also relates to a use of an implantation device according to the invention for the implantation of stents, self-expanding stents, pharmaceutical-agent-releasing stents, and/or biodegradable stents, as well as for the treatment of stenoses.

### BRIEF DESCRIPTION OF THE DRAWINGS

FIG. 1 illustrates an exemplary embodiment of an implantation device according to the invention in the form of a balloon catheter.

FIG. 2 illustrates another exemplary embodiment of an implantation device according to the invention for implanting self-expanding stents.

### DETAILED DESCRIPTION OF EXAMPLE EMBODIMENTS

The invention is described below in more detail based on the exemplary embodiments.

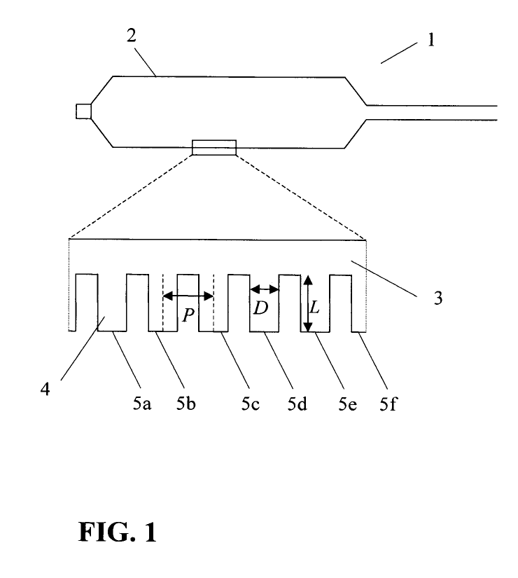

The embodiment shown in FIG. 1 of the implantation device according to the invention is a balloon catheter 1. Balloon catheter 1 has a dilatable section 2. A plurality of hair-like extensions 4 are located on at least one part of the outwardly-facing surface 3 of dilatable section 2. After attachment or crimping of the stent to be implanted onto the stent to be implanted, this outwardly-facing surface 3 can be brought into contact with an inner surface of the stent and functions to affix the stent on the implantation device. Hair-like extensions 4 have a defined mean diameter D, a defined mean length L, and a defined averaged center-to-center distance P. Here D, L, and P are selected such that van der Waals forces are created between hair-like extensions 4 and an inner surface of a stent to be implanted, which forces allow for fixation of the stent on the implantation device. Each of hair-like extensions 4 has a surface that is contactable with an inner surface of a stent to be implanted, illustrated by way of example in 5a through 5f.

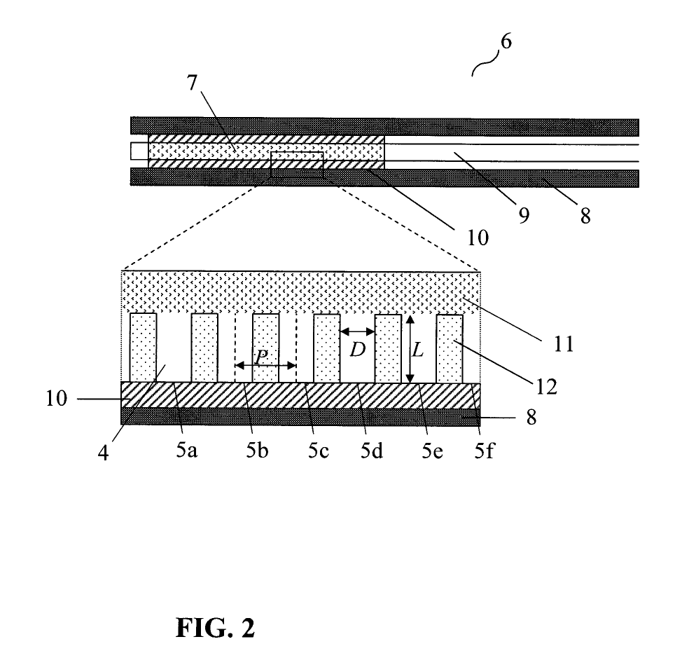

In FIG. 2, an implantation device 6 for implanting self-expanding stents is illustrated by way of example as another embodiment according to the invention. Device 6 has an interior tube 9, a retractable exterior tube 8, as well as a section 7 that is provided for the attachment or crimping of the stent to be implanted. Exterior tube 8 is designed such that it can be retracted relative to the interior tube, thereby enabling section 7 to be exposed. In section 7, device 6 has a self-expanding stent 10 that is affixable in section 7 between interior tube 9 and exterior tube 8. A plurality of hair-like extensions 4 is located at least on one part of the outwardly-facing surface 11 of section 7. This outwardly-facing surface 11 is at least partially in contact with an inner surface of stent 10 and functions to affix stent 10 on implantation device 6. Hair-like extensions 4 have a defined mean diameter D, a defined mean length L, and a defined averaged center-to-center distance P. Here D, L, and P are selected such that van der Waals forces are created between hair-like extensions 4 and an inner surface of a stent 10 to be implanted, which forces allow for fixation of stent 10 on implantation device 6. Hair-like extensions 4 each have a surface that is contactable with an inner surface of a stent 10 to be implanted, as illustrated by way of example in 5a through 5f. A lumen 12 is located between adjacent hair-like extensions 4. Contact between stent 10 and hair-like extensions 4 of section 7 results in a fixation of the stent on implantation device 6 as long as the stent continues to be covered by parts of retractable exterior tube 8. Once implantation device 6 has been positioned such that stent 10 can be implanted at the desired location, exterior tube 8 is retracted, stent 10 is exposed, detaches due to inherent radial forces from section 7 of interior tube 9, and expands automatically until it has, for example, become attached to a vascular wall and remains in position there. After implantation has been completed, device 6 can be removed, now without implanted stent 10.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

### LIST OF REFERENCE NOTATIONS

1 balloon catheter
2 dilatable section
3 part of the outwardly-facing surface of dilatable section 2
4 hair-like extension
5a through 5f surface of the hair-like extension that is contactable with an inner surface of the stent to be implanted
6 implantation device for implanting self-expanding stents
7 section which is provided for attaching or crimping the stent to be implanted
8 retractable exterior tube
9 interior tube
10 self-expanding stent
11 outwardly-facing surface of section 7
12 lumen between adjacent hair-like extensions

1. Implantation device for stents, which device has hair-like extensions, at least on one part of an outwardly-facing surface of a section of the device, which section is provided for the attachment or crimping of the stent to be implanted, wherein the mean diameter D, the mean length L, and averaged center-to-center distance P of the hair-like extensions relative to each other are selected such that a stent is essentially affixable by van der Waals forces on the surface covered with hair-like extensions.

2. Implantation device according to claim 1, wherein the implantation device comprises a catheter.

3. Implantation device according to claim 1, wherein the implantation device has a dilatable balloon surface onto which a stent can be crimped.

4. Implantation device according to claim 1, wherein the implantation device has a surface for implantation of self-expanding stents.

5. Implantation device according to claim 1, wherein the hair-like extensions are essentially cylindrical.

6. Implantation device according to claim 1, wherein D is selected from a range between 0.05 and 5 μm.

7. Implantation device according to claim 1, wherein L is selected from a range between 0.1 and 20 μm.

8. Implantation device according to claim 1, wherein P is selected from a range between 0.1 and 5 μm.

9. Implantation device according to claim 1, wherein the ratio of D to L is selected from a range between 0.5 and 2.

10. Implantation device according to claim 1, wherein the ratio of P to D is selected from a range between 1 and 50.

11. Implantation device according to claim 1, wherein the hair-like extensions are coated with a polymer having catechol side groups.

12. Implantation device according to claim 11, wherein the coating has a thickness of less than 100 nm.

13. Implantation device according to claim 1, wherein the implantation device additionally has an implantable stent, an implantable pharmaceutical-agent-releasing stent, or an implantable biodegradable stent.

14. A method for implanting stents comprising the step of using an implantation device according to claim 1 to effect implantation of stents, self-expanding stents, pharmaceutical-agent-releasing stents, and/or biodegradable stents.

15. A method for treating stenoses comprising the step of using an implantation device according to claim 1.

16. Implantation device according to claim 1, wherein P is selected from a range between 0.5 and 3 μm.

17. Implantation device according to claim 1, wherein the ratio of D to L is selected from a range between 0.75 and 1.5.

18. Implantation device according to claim 1, wherein the ratio of P to D is selected from a range between 1.5 and 10.

19. Implantation device according to claim 11, wherein the coating has a thickness of between 5 nm and 50 nm.

20. An implantation device for stents comprising:
a section of the device configured for attachment to the stent to be implanted, the section having an outwardly facing surface;
substantially cylindrical extensions on at least a portion of the outwardly-facing surface, the extensions having a mean diameter D between about 0.05 and about 5 μm., a mean length L between about 0.1 and about 20 μm, and an average center-to-center distance P between about 0.1 and about 5 μm, wherein the ratio of D to L is between about 0.75 and 1.5, the ratio of P to D is between about 1 and 50; and,
a polymer coating on the extensions having a thickness of less than about 100 nm and having catechol side groups.
